(19) United States
(12) Patent Application Publication
(10) Pub. No.: US 2009/0234296 A1
(43) Pub. Date: Sep. 17, 2009

(54) Protective device for an intravenous catheter or other peripheral body penetrating object

(76) Inventor: Carroll V. Robison, Lindale, TX (US)

Correspondence Address: RONALD B. SEFRNA, P.O. BOX 567, TYLER, TX, 75710, US

(21) Appl. No.: 12/214,470

(22) Filed: Jun. 19, 2008

Related U.S. Application Data
(60) Provisional application No. 60/936,209, filed on Jun. 20, 2007.

(51) Int. Cl. A61M 5/14

(52) U.S. Cl. 604/179

(57) ABSTRACT

A device for protecting and cushioning the site of a penetration in a patient's limb and the external portion of a catheter or other medical device inserted partially through the penetration includes a preferably soft and flexible cover to be wrapped around the limb in which the penetration is made over the penetration and catheter, a stiff but deformable stabilizer band disposed within the cover, to be wrapped around the limb with the cover, the stabilizer band to provide protection and cushioning without a propensity to roll along the patient's limb, and fastening means for releasably connecting the device around a patient's limb.

### RELATED APPLICATION DATA-CLAIM TO PRIORITY

This application claims the priority benefits of U.S. Provisional Patent Application Ser. No. 60/936,209, filed Jun. 20, 2007, titled “Protective Device For Intravenous Catheter”.

### FIELD OF THE INVENTION

The present invention generally relates to devices for protecting and stabilizing the external portion of a medical device extending through a penetration in a body part of a patient, and in its preferred embodiments more specifically relates to a device for protecting and cushioning the external portion of a peripherally inserted central catheter placed through penetration in a limb of an ambulatory human patient.

### BACKGROUND

In the course of long term drug treatment, including but not limited to chemotherapy for cancer patients, it is a relatively common practice to fit the patient with a central catheter for the administration of the drug therapy. A central catheter may remain in place in the patient's body for an extended period of time without damage, allowing the patient to remain ambulatory between treatments and to leave the hospital or other treatment center with the catheter in place. Historically, central catheters were inserted through the chest wall, but it has become increasingly common for a central catheter to be inserted peripherally, typically in a patient's upper arm, and routed through a vein to the chest, so that the drug is administered into a central vein. Whether centrally inserted or peripherally inserted, a portion of the catheter remains disposed outside the patient's body, and the external portion of the catheter should be shielded and protected for the patient's health and safety, and for the patient's comfort.

A common approach to protecting the external portion of the catheter, which, for convenience, is referred to in this application as the catheter, is to wrap an elastic bandage around the patient's arm or other insertion location in the body to secure the catheter between the patient's body and the wrapped bandage. Although a wrapped flexible elastic bandage can be effective in protecting the catheter and shielding it from contact, the elastic bandage approach has a number of disadvantages. An elongate elastic bandage can be difficult for the patient to wrap and secure, especially around an arm. The bandage can also be difficult to adjust during use. Wearing an elastic bandage for an extended period of time can also become very uncomfortable for the patient. Further, especially for a heavy patient, an elastic bandage has a tendency to roll and bunch as the patient moves, making the bandage even more uncomfortable.

Devices for stabilizing a peripheral intravenous catheter, which is left in place only while the patient is in the treatment facility, during administration of drugs or other fluids are known in the prior art, but such devices are neither designed for nor suitable for long term use with an ambulatory patient. There remains a need for an effective, comfortable, easily positioned and removed, and inexpensive device for protecting especially a peripherally inserted central catheter between treatments, and over an extended period of time.

### SUMMARY OF THE INVENTION

The present invention provides a protective wrap device for securing, protecting, and cushioning a catheter disposed in a patient's body. The device is formed as an elongate, generally flat body with a preferably soft, breathable fabric cover. In the preferred embodiment of the device, one or more strips of semi-flexible material are disposed within the cover, to provide a degree of rigidity or stiffness to the device so as to prevent it from rolling up or down on the patient during use. The inside face of the cover of the device may be provided with a flexible pocket into which the catheter is inserted as the device is positioned and wrapped, to retain and secure the catheter itself and assure that all parts of the catheter remain covered by the device. The device is secured around the arm or other body part of a patient by, preferably, hook and loop fasteners, commonly identified by the trademark “Velcro”; so that a comfortable but secure, and adjustable fit can be readily achieved, and to facilitate placement of the device by the patient without assistance.

The structure and features of the device of the invention will be described in detail with reference to the accompanying drawing figures.

### BRIEF DESCRIPTION OF THE DRAWINGS

FIG. 1 is a top plan view of the preferred embodiment of the device of the invention, showing the inner face of the device.

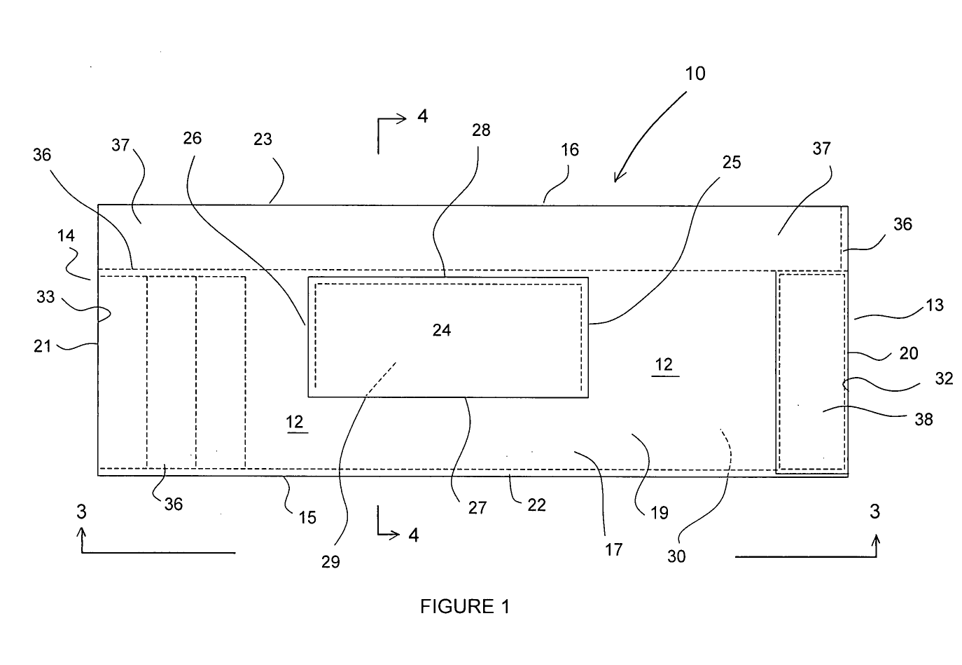

FIG. 2 is a bottom plan view of the preferred embodiment of the device of the invention, showing the outer face of the device.

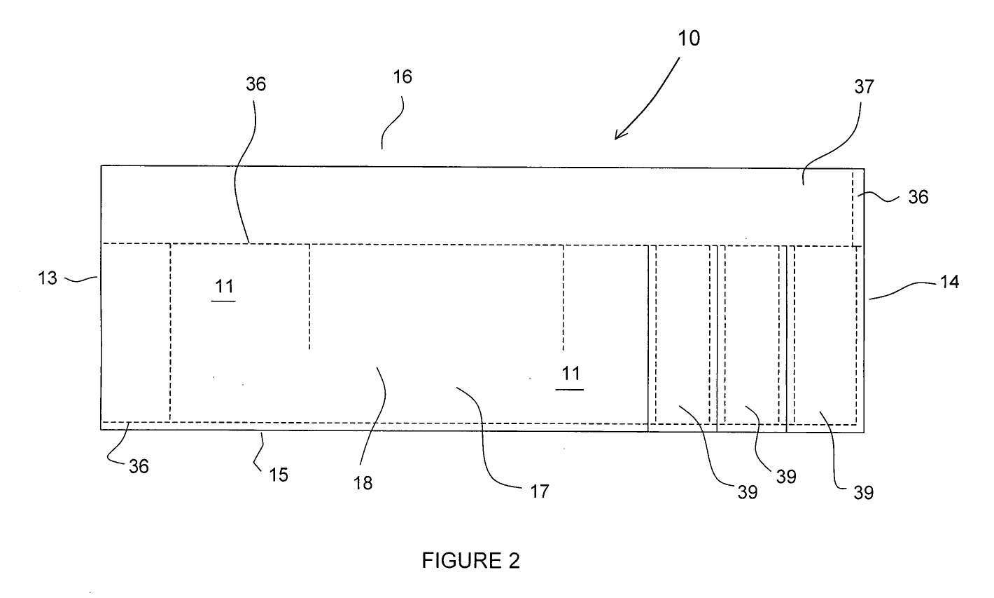

FIG. 3 is an edge view of the preferred embodiment of the device of the invention, along line 3-3 of FIG. 1.

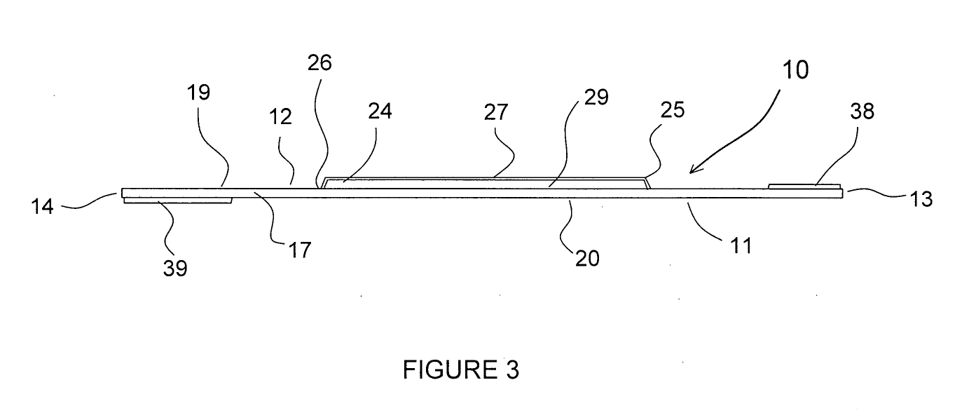

FIG. 4 is a sectioned end view of the preferred embodiment of the device of the invention, along line 4-4 of FIG. 1.

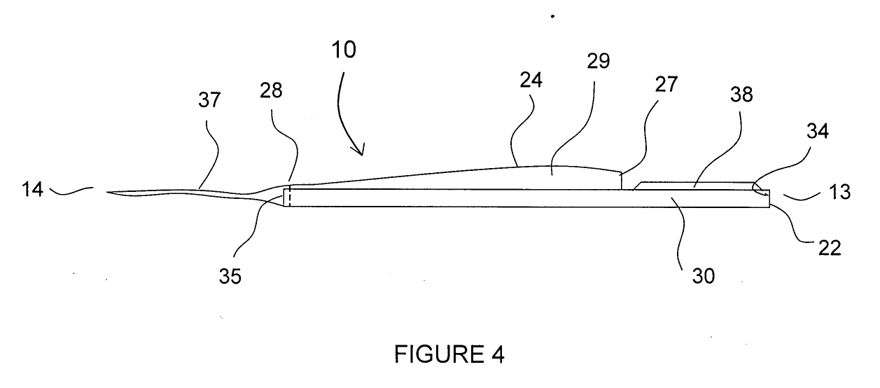

FIG. 5 is a top plan view of the inner face of a variation of the device of the invention without a catheter pocket.

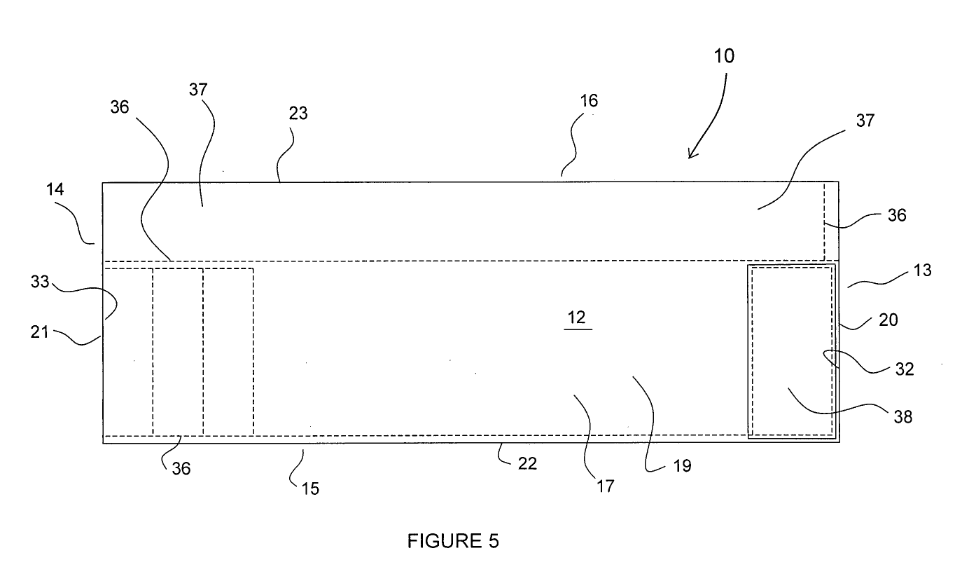

FIG. 6 is a top plan view of the inner face of an alternative embodiment of the device of the invention.

FIG. 7 is a partially cut-away top plan view of the inner face of another alternative embodiment of the device of the invention.

FIG. 8 is an illustration of the device of the preferred embodiment of the invention in place upon the arm of a patient, showing one manner of use of the device.

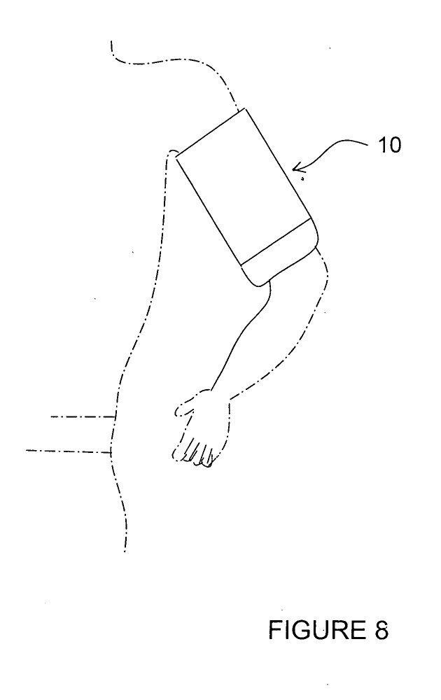

### DESCRIPTION OF THE INVENTION

In the preferred embodiment, the device of the invention, generally identified by reference number 10, comprises an elongate semi-flexible generally planar body with an outer face 11, an inner face 12, a first end 13, a second end 14, a first edge 15, and a second edge 16. It is preferred that the device be formed with a generally rectangular configuration, with generally square corners at the intersections of the ends and edges, but it is to be understood that the device could be formed in other configurations, such as but not limited to an elongate oval, within the scope of the invention.

The device 10 includes a cover 17, preferably formed of a flexible, soft, breathable fabric, to facilitate conforming the body of the device to the portion of the body a user in the location of the catheter to be protected, and for the comfort of the user. Cover 17 includes an outer face 18, generally coextensive with outer face 11 of device 10, an inner face 19, generally coextensive with inner face 12 of the device, a first end 20, a second end 21, a first edge 22, and a second edge 23. The ends and edges of the cover 17 are generally coextensive with the ends and edges of the device.

In one embodiment of the device, a catheter pocket 24 is connected to the inner face 19 of cover 17, to receive and protect the external portion of a catheter during use of the device. In this embodiment, pocket 24 is formed in a generally rectangular configuration, with a first end 25, a second end 26, a first edge 27, and a second edge 28, and is connected to the inner face 19 of the cover along the first and second ends and the second edge of the pocket, leaving first edge 27 open toward second edge 23 of the cover, for access to the interior 29 of the pocket. It is preferred that pocket 24 be formed of the same fabric material as cover 17, and is connected to cover 17 by stitching. It is also preferred that pocket 24, if used, be generally centered between first and second edges 22 and 23 of the cover, and offset slightly toward second end 21 of the cover rather than being centered between the first and second ends of the cover.

Pocket 24 is preferably formed from a single sheet of material, generally twice the size of the completed pocket, that is folded over itself into a double layer of material. The free ends and edge of the folded over piece of material are connected to cover 17, leaving the folded edge without connection to the cover as open edge 27 of the pocket. It is to be understood, however, that neither the material of construction nor the folded construction of pocket 24 is critical, and if used, pocket 24 may be formed of other materials and with, for example, a single layer construction, if desired, within the scope of the invention.

Cover 17 is preferably formed from a single sheet of fabric that is folded over itself. The free ends and edge of the folded sheet are connected together, preferably by stitching, to form the cover. In the preferred embodiment, and as shown in the drawing figures, the connected free edges of the sheet form first edge 22 of cover 17, with the folded edge of the sheet forming second edge 23 of the cover.

The device of the invention further includes a stabilizer means for maintaining the position of the device in place on a patient's limb, without sliding and without rolling up upon itself as a result of the patient's movements. In the preferred embodiment, the stabilizer means comprises a single stabilizer band 30 disposed and connected within a substantial portion of the interior 31 of cover 17, between the inner and outer faces of the cover. Stabilizer band 30 is preferably a flat, relatively thin generally planar band formed of a semi-flexible shape retentive material with sufficient flexibility to allow the band to be readily rolled, bent, or folded, but that will return to its original planar configuration when released. A non-woven material commercially available under the brand name “Polartec®” has been found to be a well suited material for the construction of stabilizer band 30, but it is to be understood that the scope of the invention is not limited to this particular material, or to any other particular material, for the formation of band 30, and that any material with suitable characteristics may be used. It is preferred that the material of construction of band 30 be permeable to air and moisture, washable, hypo-allergenic, and with sufficient flexibility to closely conform to the shape of an arm or other body part when wrapped around the body part. The material of construction of band 30 should also retain its original configuration and properties, e.g., stiffness, after repeated washing and drying. As non-limiting examples, it is contemplated that materials commonly used as stabilizers or stiffeners in sewing would be suitable for use as band 30, and that other non-textile materials, such as an open cell or closed cell foam, may also be used.

In the preferred embodiment, stabilizer band 30 extends the full length of the device, between the first and second ends 20 and 21 of cover 17, with first end 32 of band 30 aligned with first end 20 of the cover and with second end 33 of band 30 aligned with second end 23 of the cover. Stabilizer band 30 also extends from the first edge 22 of the cover, with the first edge 34 of band 30 aligned with first edge 22 of the cover through at least a majority of the distance between first edge 22 and second edge 23 of the cover. In one embodiment of the device, second edge 35 of band 30 does not extend fully to the second edge 23 of the cover. In forming the device, stabilizer band 30 is preferably placed and positioned in the interior of cover 17 before the edges of the sheet of material forming the cover are stitched together, and the first and second ends and the first edge of band 30 are preferably caught by the line or lines of stitching 36 used to close the cover, so as to assure that the stabilizer band will remain in the desired disposition within the cover. It is also preferred that a line of stitching 36 be formed between the first and second ends of the cover and stabilizer band along the second edge of the stabilizer band, extending though the cover and the stabilizer band to secure the second edge of the stabilizer band. Although it is preferred that cover 17 and stabilizer band 30 be permanently secured together, it is to be understood that the stabilizer band may be disposed in the interior of the cover without connection to the cover. If at least one end or one edge of the cover is releasably connected, so as to allow the cover to be opened, the stabilizer band would then be removable from the cover, allowing, e.g., each to be replaced without replacing the other.

The portion of cover 17 between the second edge of stabilizer band 30 and the second edge of the cover, through which stabilizer band 30 does not extend, forms skirt 37 that extends loosely from the stabilized portion of the body of the device. Skirt 37 provides some additional cushioning along the second edge of the device for the comfort of the user. However, it is to be understood that skirt 37 may be omitted within the scope of the invention, with stabilizer band 30 extending fully between the first and second edges of the cover.

In the preferred sequence of construction, catheter pocket 24, if used, is connected to cover 17 after the stabilizer band has been positioned within cover 17 and the ends and edge of those components secured together. It is preferred that second edge 28 of pocket 24 be aligned with the second edge 35 of stabilizer band 30, whether skirt 37 is present or not. It is also preferred that the line(s) of stitching 36 used to connected pocket 24 to cover 17 extend fully through the inner and outer faces of the cover as well as through stabilizer band 30. It is preferred that cover 17 and stabilizer band 30 be connected along at least the ends and edges of the stabilizer band, and additional lines or points of connection between the cover and the stabilizer band may certainly be used to help prevent shifting of the cover and/or stabilizer band relative to each other.

The device of the invention is intended to be used to protect and cover the external portion of an intravenous catheter by placing the external portion of the catheter in pocket 24 of the device, if the pocket is used or otherwise under and fully covered by the second edge of the device, wrapping the device around the part of the body in which the catheter is positioned, and then securing the device around the body part with releasable fastening means to prevent it from unwrapping. In the preferred embodiment the fastening means is a two component hook and loop type fastener, commonly referred to by the brand name “Velcro®”. A strip 38 of the hook component of the fastener is disposed on the inner face 19 of cover 17 at first end 20, extending between the first edge 22 of the cover and the second edge 35 of stabilizer band 30, and also extending from first end 20 of the cover a short distance toward the second end of the cover. A plurality of strips 39 of the loop component of the fastener, each preferably of the same dimension as strip 38, are disposed on the outer face 18 of cover 17, extending between the second edge 32 of the cover and second edge 35 of the stabilizer band, and from second end 21 of the cover toward the first end of the cover. Strip 38 and strips 39 are preferably connected by stitching 36 extending through the fastener strips, both faces of the cover, and the stabilizer band. Extending the stitching through the entire structure when connecting the components of the device as described services to secure the stabilizer band within the cover at several locations along the length of the device, and prevents the stabilizer band from shifting within the interior of the cover. Although the disposition and connection of strips of the hook and loop components as described are preferred, it is to be understood that variations in the hook and loop fasteners could be made, such as, without limitation, using a single piece of the loop component rather than a plurality of strips of that component. It is preferred that the loop component extend a greater distance from the second end of the body of the device toward the first end than the hook component extends from the first end toward the second end, so that the points of connection between the hook component and the loop component, and thus the fit of the device around a limb can be adjusted.

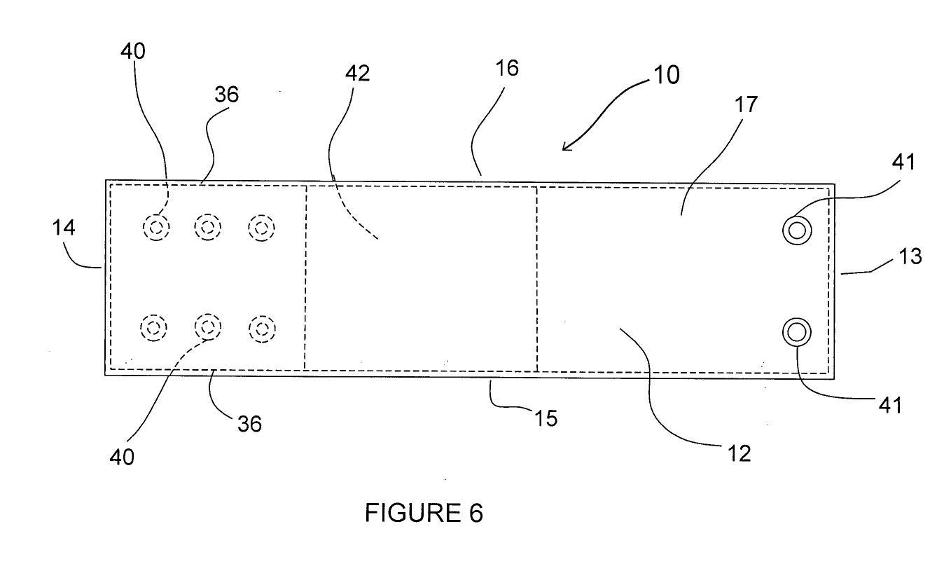

It is also to be understood that though the use of hook and loop fasteners is preferred, that other types of fastening means could be used within the scope of the invention. As one non-limiting example, illustrated in FIG. 6, snaps may be used, preferably with a plurality of female snap components 40 arrayed along a portion of the length of the device and connected to the outer face, to provide multiple choices for connection of male snap components 41 disposed near the first end of the device and connected to the inner face. As further examples, straps and buckles could be used, as could elastic bands placed over and around the device when in place on a limb, though with less convenience than the preferred hook and loop fastener means.

It is contemplated that the device of the invention will be most often used to protect and cover an intravenous catheter inserted in a patient's upper arm. As briefly described above, the device is used in that configuration by placing the device on the patient's arm with the first edge of the body adjacent to the shoulder, and with skirt 37 adjacent to the elbow. In an embodiment with a pocket 24, the external portion of the catheter is placed into the pocket through the open end of the pocket, and arranged in the pocket so that the catheter is in a reasonably flat and comfortable position. It is contemplated that the use of a pocket may help stabilize the catheter, and will also prevent direct contact between the catheter and the patient's skin, which often becomes very sensitive during the course of treatment. In an embodiment without a pocket, the device is positioned on the patient's arm over the catheter and the second end of the device is placed along and partially wrapped around the arm and held in position while the first end of the device is wrapped around the arm and releasably connected to the outer surface of the device using the selected fastening means. For proper fit, the length of the device, between its first and second ends, should be sufficiently greater than the circumference of the arm, or other body part, around which the device is to be wrapped and secured, to provide a sufficient overlap for engagement and adjustment of the fastening means without creating enough bulk to be uncomfortable. Accordingly, it is contemplated that the device of the invention will be provided in a variety of lengths, to accommodate patients of different sizes. It is also contemplated that the device may be provided with differing widths, between the edges of the device, to accommodate arms of different lengths. Because the device is readily sizable and adaptable, it may be effectively used by patients ranging from young children to large adults.

The device is wrapped tightly enough to prevent it from sliding over the elbow and off the arm, but need not be wrapped so tightly as to cause discomfort for the patient. The inclusion of skirt 37, which is disposed adjacent to the user's elbow, provides additional cushioning at that edge of the wrapped device and helps prevent constriction of the arm when the elbow is flexed. With the device in place on a user's arm the stabilizer band 30 performs at least two functions that are significant to the effectiveness of the device. In one function, the stabilizer band prevents the wrapped device from rolling on the patient's arm, either from the shoulder toward the elbow, or from the elbow toward the shoulder. Preventing the device from rolling not only assures that the catheter is properly protected and secured, but also substantially enhances the patient's comfort. In addition, because the stabilizer band is relatively stiff, it assists in retaining the device in place on the patient's arm, and reduces the tendency of any device wrapped around an upper arm to slide downward over the elbow.

In an alternative embodiment, illustrated in FIG. 6, stabilizing band 30, which extends fully between the ends of the device in the preferred embodiment and wraps fully around the limb during use of the preferred embodiment can be replaced by a stabilizing pad 42 of shorter length. In this embodiment, the stabilizer pad is disposed within the portion of the hollow interior of cover 17 between its first and second ends that will overlie the catheter penetration site and external portion of the catheter, and is connected to the cover as described above. In a variation of this embodiment, cover 17 may be formed as a single layer (eliminating the hollow interior), and the stabilizer pad, preferably wrapped in its own cover, attached to the single layer cover in the desired location. This embodiment further reduces the bulk of the device on a patient's arm by eliminating a portion of the bulk of the stabilizer. However, if this alternative device rotates on a patient's arm during use the stabilizer pad may move from over the catheter and negate much of the advantage the stabilizer provides. Therefore, if this alternative embodiment is used consideration should be given to using a slip-resistant material for cover 17, and/or making pad 42 with sufficient area to remain over the catheter with limited rotation of the cover on the patient's arm or other limb.

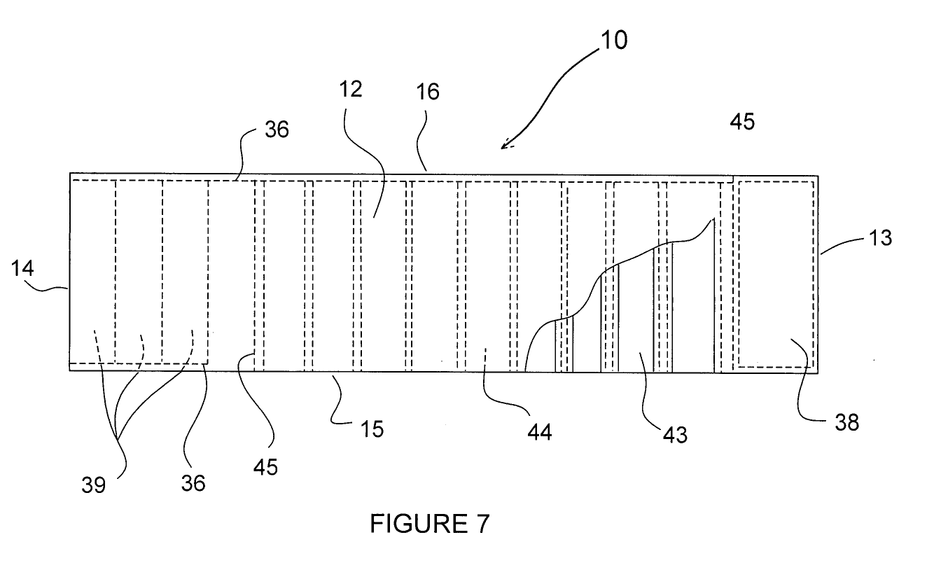

In another alternative embodiment, shown in FIG. 7, single stabilizer band 30 is replaced by a plurality of narrow generally planar slats 43 arrayed along the length of the device with each disposed adjacent to each other in parallel relation, and perpendicular to the edges of the device. Each slat 43 is received within an open ended sleeve 44 formed in cover 17, each of which extends from the first edge of the cover toward the second edge. Sleeves 44 are preferably formed by connecting the two layers of cover 17 together with spaced apart parallel connection lines 45 extending from the first edge of the cover toward the second edge, running from the second end of the cover over the majority of the distance to the first end, with each connection line connecting both layers of the cover. In this embodiment the two layers of the cover at the first edge are open between the connection lines 45, to form the open ends of the sleeves. The open ends of sleeves 44 may be left open after slats 43 are inserted, allowing removal of the slats, or maybe connected after insertion to close the sleeves with the slats contained therein.

This alternative embodiment is used in essentially the same way as the other embodiments, and is positioned around a patient's arm or other body part as described above. Although slats 43 are separated from each other, they are immediately adjacent to each other and provide what is effectively a continuous stabilizer to protect and cushion the catheter and insertion site. The only significant difference from the single stabilizer band embodiment is that the stabilizer slats extend along a patient's limb rather than around the limb when the device is in use.

The device of the invention, in all embodiments and variations, is more comfortable for a patient and is more easily adjustable during use than, e.g., a wrapped elastic bandage. The device of the invention is wrapped around the arm in a single layer with only a slight overlap of the ends of the device, whereas an elastic bandage is typically wrapped around the arm in multiple layers. Adjusting the fit of an elastic bandage requires removing a substantial part of the bandage and fully re-wrapping it. Adjusting the fit of the device of the invention is easily accomplished by releasing the fastener and then re-fastening it to be slightly tighter or looser, as desired, without unwrapping or removing the device.

The foregoing description of the preferred embodiment of the device of the invention, and of certain variations within the scope of the invention is intended to be illustrative and not limiting. The device is susceptible to other alternative embodiments and variations within the scope of the invention, which is intended to be broadly construed.

1. A protective device for protecting the site of a peripheral human body penetration and protecting the external portion of a medical device extending through the penetration with a portion of the medical device disposed within the body and a portion disposed externally of the body, comprising,
a cover to be placed over the penetration site and the external portion of the medical device, and releasably fastened around the body part in which the penetration is made, said cover having a first end and a second end, a first edge and a second edge, and an outer face and an inner face, and being of a length between said first and second ends greater than the circumference of the body part in which the penetration is made so as to allow said cover to be wrapped fully around the body part;
a stabilizing means disposed within said cover for the purpose of stiffening said cover so as to substantially maintain the position of said cover over the penetration site on the body part in which the penetration is made, and so as to substantially prevent each of said edges of said cover from rolling toward the opposite edge during use; and
fastening means for releasably connecting said cover to itself after said cover is wrapped around the body part in which the penetration is made.

2. The protective device of claim 1, wherein said cover comprises a strip of a soft, flexible, washable, hypo-allergenic material permeable to air and moisture, said strip having a first end and a second end, and a first edge and a second edge, said strip folded over itself with said ends and edges respectively aligned with each other and connected so as to form a two layer cover with a hollow interior.

3. The protective device of claim 1, wherein said cover comprises two strips of a soft, flexible material, each having a first and second end and a first and second edge, one of said strips disposed on top of the other of said strips with said respective first ends, second ends, first edges, and second edges of said strips aligned, said strips connected together adjacent to said aligned ends and edges to form a two layer cover with a hollow interior.

4. The protective device of claim 1, wherein said cover includes a loose skirt extending outwardly from said second edge and between said first and second ends.

5. The protective device of claim 1, wherein said stabilizing means comprises a generally planar piece of a stiff, shape retentive material with sufficient flexibility to be wrapped around the body part in which the penetration is made.

6. The protective device of claim 1, wherein said fastening means comprises a two part releasable hook and loop material, one of said parts of said material connected to said cover on said outer face thereof, and the other of said parts of said material connected to said cover on said inner face thereof, disposed such that said parts are brought together when said connected cover and stabilizing means are wrapped around the body part in which the penetration is made.

7. The protective device of claim 1, wherein said cover further includes an open edged pocket connected to said inner face of said cover between said first and second ends and said first and second edges of said cover with said open end of said pocket facing toward said second edge of said cover and disposed adjacent to said second edge of said cover, said pocket being configured and dimensioned to receive within said pocket the external portion of the medical device to be protected when the device is positioned on the body part in which the penetration is made.

8. The protective device of claim 1, wherein said cover is formed as a two layer structure, wherein said stabilizing means is disposed between said layers, and wherein said stabilizing means is connected within said cover by stitching extending through both of said layers of said cover and said stabilizing means.

9. The protective device of claim 1, wherein said cover is formed as a two layer structure with a hollow interior between said layers, and wherein said stabilizing means is disposed in said interior of said cover without connection between said cover and said stabilizing means.

10. The protective device of claim 1, wherein said stabilizing means is removably connected within said cover.

11. The protective device of claim 1, wherein said stabilizing means is permanently connected within said cover.

12. The protective device of claim 1, wherein
said cover is formed as a two layer structure, said layers connected along said first and second ends and along a connection line adjacent to said second edge, and said layers connected to each other between said first edge and said connection line at spaced intervals between said first and second ends to form a plurality of sleeves extending from said first edge toward said second edge in generally perpendicular relation to said edges; and
wherein said stabilizer means comprises a plurality of generally planar slats formed of a stiff, deformable, and shape retentive material, each of said slats configured and dimensioned to be received in one of said sleeves and extend from closely adjacent to said first edge to said connection line between said layers of said cover.

13. A protective device for covering, protecting, and cushioning the site of and the external portion of a medical device inserted through a penetration in a limb of a patient, with an internal portion of the medical device disposed within the patient's body and an external portion disposed externally of the patient's limb, comprising,
a cover to be placed over the penetration site and the external portion of the medical device, and wrapped and releasably fastened around the limb in which the penetration is made, said cover having a first end and a second end, a first edge and a second edge, and an outer face and an inner face, said cover formed of a soft, flexible material as a two layer structure with a hollow interior between said layers, said layers connected adjacent to said ends thereof and adjacent to at least one of said edges, said cover having a length between said first and second ends of greater magnitude than the circumference of the limb in which the penetration is made so as to allow said cover to be wrapped fully around the limb with said inner face of said cover received against the limb;
a pocket connected to said inner face of said cover adjacent to said second edge of said cover, said pocket having one open edge free of connection to said cover, said open edge facing toward said second edge of said cover, said pocket configured and dimensioned, and disposed on said inner face of said cover so as to receive the external portion of the medical device when said cover is positioned to be wrapped around the limb in which the penetration is made;
stabilizing and cushioning means disposed in said hollow interior of said cover and extending within said hollow interior through substantially the full length of said cover between said first and second ends, and extending between said first and second edges, for the purpose of stiffening said cover so as to substantially maintain the position of said cover over the penetration site in the limb in which the penetration is made, and so as to substantially prevent each of said edges of said cover from rolling toward the opposite edge during use; and
fastening means for releasably connecting said inner face of said cover at said first end thereof to said outer face of said cover after said cover is wrapped around the limb over the penetration site and the external portion of the medical device.

14. The protective device of claim 13, wherein said stabilizing means comprises a single band of material, said band to wrap circumferentially around the limb in which the penetration is made.

15. The protective device of claim 14, wherein said band is connected to said cover so as to maintain the position of said band in said interior of said cover.

16. The protective device of claim 13, wherein
said layers of said cover are connected along said first and second ends and along a connection line adjacent to said second edge, and said layers are connected to each other between said first edge and said connection line at spaced intervals between said first and second ends to form a plurality of sleeves extending from said first edge toward said second edge in generally perpendicular relation to said edges; and
wherein said stabilizer means comprises a plurality of generally planar slats formed of a stiff, deformable, and shape retentive material, each of said slats configured and dimensioned to be received in one of said sleeves and extend from closely adjacent to said first edge to said connection line between said layers of said cover, such that each of said slats extends along the limb when said cover is wrapped around the limb.

17. The protective device of claim 13, wherein said cover and said stabilizer means are formed of materials that are hypo-allergenic, washable and reusable, and permeable to air and moisture.

18. The protective device of claim 13, wherein said fastening means comprises a two part hook and loop fastener with a first part and a second part, said first part connected to said inner face of said cover at said first end thereof between said first and second edges, and said second part connected to said outer face of said cover between said first and second edges, said second part disposed such that said first part will overlie and connect to said second part when said cover is wrapped around the limb in which the penetration is made.

19. A method of protecting and cushioning the site of a penetration in the limb of a human patient and the external portion of a medical device extending through the penetration with an internal portion of the medical device in the interior of the limb and an external portion of the medical device disposed on the exterior of the limb, comprising the steps of,
forming a protective device having a soft flexible cover with first and second ends, first and second edges, and an inner face and an outer face, the cover configured and dimensioned to be wrapped around the limb over the site of the penetration and external portion of the medical device without interfering with the joints of the limb, and having a stabilizer formed of a relatively stiff but deformable and shape retentive material, disposed within the cover and extending substantially between the first and second ends thereof and substantially between the first and second edges thereof;
positioning the protective device on the limb with a part of the inner face of the cover and a part of the stabilizer directly overlying the penetration site and the external portion of the medical device;
wrapping the protective device around the limb in which the penetration is made, with the cover and the stabilizer fully covering the penetration site and the external portion of the medical device; and
releasably connecting the protective device so as to retain it around the limb to protect and cushion the penetration site and external portion of the medical device without allowing movement or deformation of the device sufficient to expose any part of the penetration site or medical device, thereby allowing the patient to move and use the limb without interference or disruption of the penetration site and the external portion of the medical device.

20. The method of claim 18, further comprising the additional steps of,
prior to the step of positioning the protective device on the limb, forming a pocket on the inner face of the cover, positioned so as to receive the external portion of the medical device within the pocket; and
prior to the step of wrapping the protective device around the limb, placing the external portion of the medical device within the pocket so as to separate the external portion of the medical device from the skin of the patient.
